(19) United States
(12) Patent Application Publication
(10) Pub. No.: US 2015/0351428 A1
(43) Pub. Date: Dec. 10, 2015

(54) METHODS AND COMPOSITIONS FOR ORALLY DOSING POSTNATAL SWINE

(71) Applicant: Animal Science Products, Inc., Nacogdoches, TX (US)

(72) Inventors: Ryan Izard, Nacogdoches, TX (US); Culley Wilson, Nacogdoches, TX (US)

(21) Appl. No.: 14/730,842

(22) Filed: Jun. 4, 2015

Related U.S. Application Data
(60) Provisional application No. 62/007,455, filed on Jun. 4, 2014.

(51) Int. Cl. A23K 1/18; A61K 47/32; A61D 7/00; A61K 39/02

(52) U.S. Cl. CPC A23K 1/184; A61K 39/0208; A61K 47/32; A23K 1/1893; A61D 7/00; A61K 2039/522

(57) ABSTRACT

Swine pharmaceutical delivery compositions comprising an active agent and a pharmaceutically acceptable carrier, methods of preparing and administering the compositions, and kits for preparing and using the compositions are provided.

### RELATED APPLICATIONS

This applications claims the benefit of U.S. Provisional Application No. 62/007,455, filed Jun. 4, 2014, the contents of which are incorporated herein by reference in their entirety.

### STATEMENT REGARDING FEDERALLY FUNDED RESEARCH

None.

### REFERENCE TO SEQUENCE LISTING

None.

### BACKGROUND

Under the conditions of modern swine husbandry, a number of beneficial products are available to improve the health and wellbeing of baby pigs. Available routes of administration of these components can be generally segmented into two phases depending on whether the pig is a newborn or after weaning.

Newborn piglets, instinctively focused on nursing, will not voluntarily eat or drink dry food, pastes or supplemental water. Consequently, beneficial products best given to piglets soon after birth are either injected or force-fed. As contemporary farms are very large, individually catching and injecting or force-feeding thousands of baby pigs daily is overly stressful to the pigs and sows, as well as being labor intensive for the farm workers.

Drinking water is not effective as a vehicle to deliver targeted oral supplements to neonatal pigs prior to weaning because the piglets merely sample water from the same supply as the mother. Consequently, additives in the water supply do not appropriately target the piglets.

Baby pigs will not voluntarily consume oral supplements until much later, and usually too late. For example, pigs require iron supplementation very early in life to overcome deficiencies in the mother's milk. Piglets, however, will not begin experimenting with dry food until they are about 14 days old, which is too late to prevent iron deficiency anemia. Yet piglets do not begin eating food in more than modest quantities until weaning.

Moreover, farmers who wish to vaccinate pigs with orally-active live vaccines or beneficial probiotics are obliged to individually force-feed the product to babies, or wait until the babies begin to wean and consume the vaccines and probiotics in their drinking water and feed. But because immunity declines as piglets start to wean, farmers often wish to administer antibiotics to compensate for declining immunity. Yet the same antibiotics inactivate bacterial vaccines or probiotics. It would therefore be beneficial to administer vaccines or probiotics prior to weaning, before antibiotics are indicated, as well as ways that encourage consumption by pre-weaning baby pigs.

### SUMMARY OF THE INVENTION

The present invention relates to novel compositions and delivery methods that are desirable to baby pigs, provide timely nutritional supplementation, support metabolic development, animal health, improved growth and feed conversion rates, and elevate humane farming practices.

In one embodiment, swine pharmaceutical delivery compositions comprising an active agent and a pharmaceutically acceptable carrier are provided. The pharmaceutically acceptable carrier may be selected from the group consisting of stabilizers, pH adjusting compositions, adjuvants, antimicrobial agents, anesthetics, corticosteroids, or a combination thereof. In some aspects, the compositions comprise a colorant, a flavor agent, or both.

In another aspect, the composition comprises an adhesion enhancing agent. In some aspects, the adhesion enhancing agent is at a final concentration from about 0.5% to 15% w/v, from about 0.5% to 10% w/v, from about 0.5% to 5% w/v, from about 0.5% to 2% w/v, and about 1.3%.

In some aspects, the adhesion enhancing agent is polyvinyl pyrrolidone or has a molecular weight of 24,000 to 90,000. In other aspects the adhesion enhancing agent is a hydrophilic polymer or copolymer that is linear or branched, crosslinked, is not biodegradable, or is selected from the group consisting of xanthan, guar, pectins, gums, guar derivatives, chitosan, dextran, maltodextrin, carrageenans, starch, polyethylene glycol, albumin, cellulose ethers, hyaluronic acid, carboxymethylhydroxyethylcellulose, hydroxypropyl cellulose, gelatins, vinyl acetates, polyvinyl pyrrolidone-vinyl acetate copolymers, polyvinyl alcohols, polyphosphoesters, N-(2-hydroxypropyl) methacrylamide (HPMA) copolymers, polyacrylic acids, polyacrylamides, polyoxazolines, divinyl ether-maleic anhydride, polyphosphazenes, including derivatives and substitutions and salts of any of the foregoing, and combinations thereof.

In another aspect, the adhesion enhancing agent is an cellulose that includes one or more of hydroxypropylmethyl cellulose (HPMC), hydroxypropyl cellulose (HPC), hydroxyethyl cellulose (HEC), and carboxy methyl cellulose (CMC), and salts thereof.

In additional aspects, the active agent comprises an ingredient selected from the group consisting of vitamin, mineral, amino acid, vaccine, and probiotic. In further aspects, the active agent comprises a peptide, polypeptide, protein, enzyme, carbohydrate, lipid, or mineral, or combination thereof. In another aspect, the active agent is selected from the group consisting of biologically viable material, a food or feed ingredient, an antimicrobial agent, an antibiotic replacement agent, a prebiotic, a probiotic, and a pharmaceutical compound. In some aspects, the pharmaceutical compound may be selected from the group consisting of analgesic, respiratory stimulant, mucolytic, and expectorant.

In some aspects, the compositions comprising an adhesion enhancing agent are viscous. In further aspects the compositions have a viscosity (cPs) of at least about 10, 20, 30, 40, 50, 60, 70, 80, 90, 100, 150, 200, 250, 300, 350, 400, 450, 500, 550, 600, 650, 700, 750, 800 cPs or higher.

In some aspects, the compositions comprising an adhesion enhancing agent further comprise a colorant, a flavor agent, or both.

In another embodiment, a method of orally dosing piglets is provided that comprises obtaining a swine pharmaceutical delivery composition, applying the composition to at least one treated pig in the vicinity of at least one piglet, and allowing the at least one piglet to voluntarily consume the composition from the at least one treated pig, wherein the at least one piglet exhibits a change in health not observed in a piglet that has not voluntarily consumed the composition.

In a further aspect, the step of applying comprises topically applying the composition, which may include by spraying, pouring, or dripping. In one aspect, the composition is topically applied to at least one teat of at least one treated pig. In another aspect, the composition comprises a vaccine. In yet another aspect, the change in health is a change in live viral titer, which may be a decrease in live viral titer that is detected three weeks after application. In some aspects, the composition in this embodiment comprises a colorant, a flavor agent, or both.

In another embodiment, a method of preparing a swine pharmaceutical delivery composition, the method comprising preparing an admixture comprising an adhesion enhancing agent, a pH adjusting agent, and a stabilizer; mixing the admixture with a solvent to form a mixture; adding at least one active agent to the mixture; and mixing the mixture to form a composition.

In some aspects the admixture is dry and powdered. In some aspects the solvent is water. In some aspects the method further comprises adding a flavoring agent, a colorant, or both, to the mixture. In some aspects, the adhesion enhancing agent is polyvinyl pyrrolidone. In some aspects, the at least one active agent is selected from the group consisting of a vitamin, mineral, amino acid, vaccine, and probiotic.

In another embodiment, a kit for preparing a swine pharmaceutical delivery composition comprising a container comprising a swine pharmaceutical delivery composition and instructions for use. In some aspects, the kit further comprises a flavoring agent and in some aspects it also comprises a colorant.

It is contemplated that any embodiment of a method or composition described herein can be implemented with respect to any other method or composition described herein.

### DETAILED DESCRIPTION

The present invention relates to compositions and methods of using the compositions to efficiently deliver active ingredients to young piglets as early as the first day of life. The delivery system and active ingredients are voluntarily consumed by litters of piglets, eliminating the need for injection syringes or specialized dosing devices, and overcoming the stress of catching, excessive handling and force-feeding individual piglets. Early voluntary consumption by newborn piglets accelerates the timeline for administration of key ingredients that, prior to the advent of this invention, were delayed until the pigs were mature enough to consume feed or drinking water. Some examples of active ingredients commonly administered include key nutrients, vaccines, beneficial bacteria, medications and antibodies against pathogens.

### DEFINITIONS

As used herein, “voluntary” and related forms refers to action initiated and carried out by an animal by its own free will. Voluntary does not include the response of an animal to physical coercion and therefore does not encompass gavage (i.e., force-feeding).

As used herein, “pharmaceutically acceptable carrier” refers to pharmaceutically acceptable ingredients such as preservatives, stabilizers, pH adjusting compositions, adjuvants, antimicrobial agents, corticosteroids, alcohols, glycols, glycerols, and glycerines, lanolin and derivatives thereof, fatty acids and derivatives thereof, fatty alcohols and derivatives thereof, and fatty esters and derivatives thereof, or combinations thereof. Pharmaceutically acceptable carriers include water, hydrophilic ointment, petrolatum, mineral oil, vegetable oil, animal oil, organic and inorganic waxes, such as microcrystalline, paraffin and ozocerite wax, natural polymers, alcohols, polyols, and the like. Additional suitable pharmaceutically acceptable carriers include, but are not limited to methyl formamide (DMF), allantoin, urazole, N,N-dimethylacetamide (DMA), dimethyl sulfoxide (DMSO), decylmethylsulfoxide, propylene glycol caprylate, polyethylene glycol monolaurate (PEGML), propylene glycol (PG), propylene glycol monolaurate (PGML), glycerol monolaurate (GML), lecithin, polyoxylglycerides, the 1-substituted azacycloheptan-2-ones, particularly 1-n-dodecylcyclazacycloheptan-2-one, alcohols, and oils safe for porcine consumption such as vegetable oils.

As used herein, “pharmaceutical” refers at least to the active agents disclosed herein.

As used herein, “acceptable carrier” refers to acceptable additional ingredients moisturizing agents or humectants, pH adjusting agents, fragrances, chelating agents, emulsifiers, thickeners, solubilizing agents, anti-irritants, colorants and surfactants.

The carrier(s) must be “acceptable” in the sense of being compatible with the other ingredients of the formulation and not deleterious to the recipient thereof.

The term “topical” refers to administration of an inventive composition at the point of application. The phrase “topically applying” refers to direct application to a surface of a pig. The composition may be applied by pouring, dropping, or spraying or by any other appropriate means.

A “pH adjusting composition” is typically added to bring the pH of the composition to the desired value. Desirable pH values are between about 6 to about 8. The compositions of the described invention therefore may be formulated to have a pH value that ranges between about 6 and about 8, or about 6.5 and about 7.5. Suitable pH adjusting agents include, but are not limited to, one or more adipic acids, glycines, citric acids, calcium hydroxides, magnesium aluminometasilicates, disodium phosphate, sodium phosphate, potassium phosphate, potassium chloride, sodium citrate, calcium lactate, sodium succinate, sodium glutamate, sodium bicarbonate, and potassium bicarbonate, and combinations thereof.

As used herein, “stabilizer” is an agent that helps stabilize the active agent in the composition. The stabilizer includes but is not limited to reducing agents. Stabilizers that may be used include sodium thiosulfate, sodium metabisulfite, sodium bisulfite, sodium sulfite, sulphur dioxide, ammonium bisulfite, and ammonium thiosulfate. Sodium thiosulfate is preferred as it possess a high neutralization ability and is considered safe and not corrosive.

Chelating agents are optionally added to the compositions of the described invention so as to enhance the preservative or preservative system. Preferred chelating agents are mild agents, such as, for example, ethylenediaminetetraacetic acid (EDTA), EDTA derivatives, or any combination thereof.

Suitable preservatives for use in the compositions of the present composition include, without limitation, one or more alkanols, disodium EDTA (ethylenediamine tetraacetate), EDTA salts. EDTA tatty acid conjugates, isothiazolinone, parabens such as methylparaben and propylparaben, propylene glycols, sorbates, urea derivatives such as diazolindinyl urea, or any combinations thereof.

As used herein “adhesion enhancing agent” refers to an agent that increases the adhesion of the compositions disclosed herein to the hair and/or skin of swine (including in particular to sows and piglets). Adhesion enhancing agents are preferably edible but not necessarily digestible or absorbable. Suitable adhesion enhancing agents include polyvinylpyrrolidone, polyvinylpyrrolidone/vinyl acetate copolymers, waxes, mineral oil, plastigel (a blend of mineral oil and polyethylene), petrolatum, white petrolatum, shellac, versagel (blend of liquid paraffin, butene/ethylene/styrene hydrogenated copolymer), polyethylene waxes, microcrystalline waxes, polyisobutene, polyvinylpyrrolidone/vinyl acetate copolymers, and insoluble polyacrylate copolymers.

Suitable adhesion enhancing agents further include xanthan, guar, pectins, gums, guar derivatives, chitosan, dextran, maltodextrin, carrageenans, starch, polyethylene glycol, albumin, cellulose ethers, hyaluronic acid, carboxymethylhydroxyethylcellulose, hydroxypropyl cellulose, gelatins, vinyl acetates, polyvinyl pyrrolidone-vinyl acetate copolymers, polyvinyl alcohols, polyphosphoesters, N-(2-hydroxypropyl) methacrylamide (HPMA) copolymers, polyacrylic acids, polyacrylamides, polyoxazolines, divinyl ether-maleic anhydride, polyphosphazenes, including derivatives and substitutions, and combinations thereof. In another aspect, the adhesion enhancing agent is a cellulose that includes one or more of hydroxypropylmethyl cellulose (HPMC), hydroxypropyl cellulose (HPC), hydroxyethyl cellulose (HEC), and carboxy methyl cellulose (CMC), and salts thereof.

As used herein “solubilizing agents” are those substances that enable solutes to dissolve. Representative examples of solubilizing agents that are usable in the context of the described invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN 80®. Other solubilizers that are usable for the compositions of the described invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

The term “colorant” also may be used in the compositions of the described invention to provide visual cues to the piglets and/or visual verification to animal caretakers that the composition is present, uniformly applied and appropriately adherent. Colorants include pigments or dyes or a combination thereof. Suitable colorants include, but are not limited to, FD&C colorants such as FD&C Blue No. 1, FD&C Blue No. 2, FD&C Green No. 3, Orange B, Citrus FD&C Red No. 2, FD&C Red No. 2, FD&C Red No. 3, FD&C Red No. 40, FD&C Yellow No. 5 and FD&C Yellow No. 6.

The term “surfactants” as used herein refers to surface-active substances, such as a detergent, emulsifiers, wetting agents, dispersants, and foaming agents. Suitable surfactants for use with the inventive compositions include, but are not limited to, sarcosinates, glutamates, sodium alkyl sulfates, ammonium alkyl sulfates, sodium alkyl ether sulfates, ammonium alkyl ether sulfates, ammonium laureth-n-sulfates, sodium laureth-n-sulfates, isothionates, glycerylether sulfonates, sulfosuccinates and combinations thereof where an anionic surfactant is desired, suitable anionic surfactants that may be used include, but are not limited to, sodium lauryl sarcosinate, monosodium lauroyl glutamate, sodium alkyl sulfates, ammonium alkyl sulfates, sodium alkyl ether sulfates, ammonium alkyl ether sulfates, and combinations thereof.

The term “flavoring agent” as used herein refers to one or more compounds or mixtures that improve the palatability and/or taste in swine. Flavoring agents include but are not limited to nutritive and non-nutritive sweeteners, flavor additives, by-products and alternative ingredients. By way of example suitable flavorants include but are not limited to sucrose, glucose, sodium saccharin, sodium cyclamate, xylitol, perillartien, sucralose, D-tryptophan, aspartame, dihydrochalcones and the like, artificial fruit flavoring (e.g., strawberry flavoring), plasma protein (e.g., spray-dried plasma protein), cheese and cheese-like flavorings, dried milk, chocolate and chocolate by-products.

The term “copolymer” as used herein is not limited to the combination of two polymers, but includes any combination of polymers. e.g., terpolymers.

As used herein, a composition is considered to be “viscous” if it has an apparent viscosity of 50 mPa*s (cP) or higher.

As used herein, “% wt/vol” means the mass-volume percentage, sometimes referred to as weight-volume percentage or percent weight per volume and often abbreviated as % m/v or % w/v, which describes the mass of the solute in g per 100 mL of the liquid. Mass-volume percentage is often used for solutions made from a solid solute dissolved in a liquid. For example, a 40% w/v sugar solution contains 40 g of sugar per 100 mL of liquid.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

As used herein, unless otherwise specified or unless the context otherwise clearly requires, “about” regarding a number or measurement means within 10% of the number or measurement.

As used herein, when the term “range” refers to integers, every integer from the minimum to the maximum values of such range is included. In addition, where multiple ranges are provided to describe a concentration or characteristic, such ranges may be combined.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All of the versions of the invention described herein are assumed to have the therapeutically effect amount(s) of constituent substances, or combinations thereof:

### DESCRIPTION

The present invention relates to compositions and methods of using the compositions to efficiently deliver active ingredients to young piglets as early as the first day of life.

In one embodiment, compositions comprising an active agent and a pharmaceutically acceptable carrier is provided. In a preferred aspect, the active agent comprises an ingredient selected from the group consisting of vitamins, minerals, vaccines, and probiotics. In another aspect, the active agent comprises an amino acid, peptide, polypeptide, protein, enzyme, carbohydrate, lipid, or mineral, or combination thereof. The active agent is preferably incorporated in the compositions at a concentration of 0.0001% to 20% by weight and more preferably 0.0001% to 5% by weight.

The active agent may also be selected from the group consisting of biologically viable material, a food or feed ingredient, an antimicrobial agent, an antibiotic replacement agent, a prebiotic, a probiotic, and a pharmaceutical compound.

In some aspects, the pharmaceutical compound may be selected from the group consisting of analgesic, respiratory stimulant, mucolytic, and expectorant.

The pharmaceutically acceptable carrier may be selected from the group consisting of stabilizers, pH adjusting compositions, adjuvants, antimicrobial agents, anesthetics, corticosteroids, or a combination thereof.

In some aspects of the invention, the compositions may comprise a pharmaceutically acceptable carrier in a final concentration (v/v) of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, or any ranges therebetween including, for example, about 5% to about 10%, about 10% to about 15%, about 12% to about 13%, about 15% to about 20%, about 20% to about 25%, about 25% to about 30%, about 30% to about 35%, about 35% to about 40%, about 40% to about 45%, and about 45% to about 50%. In another aspect, the final concentration of the pharmaceutically acceptable carrier is about 12.5% v/v.

In another aspect the composition comprises an adhesion enhancing agent. In some aspects, the adhesion enhancing agent in a final concentration (w/v) of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, or any ranges therebetween including, for example, from about 0.5% to 15% w/v, from about 0.5% to 10% w/v, from about 0.5% to 5% w/v, from about 0.5% to 2% w/v. In another aspect, the final concentration of the adhesion enhancing agent is about 1.3%.

In some aspects the adhesion enhancing agent is a hydrophilic polymer or copolymer that is linear or branched, crosslinked, is not biodegradable. Suitable adhesion enhancing agents include polyvinylpyrrolidone, polyvinylpyrrolidone/vinyl acetate copolymers, waxes, mineral oil, plastigel (a blend of mineral oil and polyethylene), petrolatum, white petrolatum, shellac, versagel (blend of liquid paraffin, butene/ethylene/styrene hydrogenated copolymer), polyethylene waxes, microcrystalline waxes, polyisobutene, polyvinylpyrrolidone/vinyl acetate copolymers, and insoluble polyacrylate copolymers, xanthan, guar, pectins, gums, guar derivatives, chitosan, dextran, maltodextrin, carrageenans, starch, polyethylene glycol, albumin, cellulose ethers, hyaluronic acid, carboxymethylhydroxyethylcellulose, hydroxypropyl cellulose, gelatins, vinyl acetates, polyvinyl pyrrolidone-vinyl acetate copolymers, polyvinyl alcohols, polyphosphoesters, N-(2-hydroxypropyl) methacrylamide (HPMA) copolymers, polyacrylic acids, polyacrylamides, polyoxazolines, divinyl ether-maleic anhydride, polyphosphazenes, including derivatives and substitutions, and combinations thereof.

One or more of hydroxypropylmethyl cellulose (HPMC), hydroxypropyl cellulose (HPC), hydroxyethyl cellulose (HEC), and carboxy methyl cellulose (CMC), and their salts, are also suitable adhesion enhancing agents.

In some aspects, the adhesion enhancing agent has a molecular weight of 24,000 to 90,000.

In some aspects, the compositions comprising an adhesion enhancing agent are viscous. In further aspects the compositions have a viscosity (cPs) of at least about 10, 20, 30, 40, 50, 60, 70, 80, 90, 100, 150, 200, 250, 300, 350, 400, 450, 500, 550, 600, 650, 700, 750, 800 cPs or higher.

In additional aspects, the active agent used in the compositions comprising an adhesion enhancing agent comprises an ingredient selected from the group consisting of vitamin, mineral, amino acid, vaccine, and probiotic. In further aspects, the active agent comprises an amino acid, peptide, polypeptide, protein, enzyme, carbohydrate, lipid, or mineral, or combination thereof. In another aspect, the active agent is selected from the group consisting of biologically viable material, a food or feed ingredient, an antimicrobial agent, an antibiotic replacement agent, a prebiotic, a probiotic, and a pharmaceutical compound. In some aspects, the pharmaceutical compound may be selected from the group consisting of analgesic, respiratory stimulant, mucolytic, and expectorant.

In an embodiment where the composition comprises a flavoring agent, the agent is selected from nutritive and non-nutritive sweeteners, flavor additives, by-products and alternative ingredients. Some preferred flavoring agents include sucrose, glucose, sodium saccharin, sodium cyclamate, xylitol, perillartien, sucralose, D-tryptophan, aspartame, dihydrochalcones and the like, artificial fruit flavoring (e.g., strawberry flavoring), plasma protein (e.g., spray-dried plasma protein), cheese and cheese-like flavorings, dried milk, chocolate and chocolate by-products. The flavoring agent is preferably incorporated in the compositions at a concentration of 0% to 2% by weight and more preferably 0.1% to 0.5% by weight.

In an embodiment where the composition comprises a colorant, a colorant such as a pigment or dye or a combination thereof may be used. Suitable colorants include, but are not limited to, FD&C colorants such as FD&C Blue No. 1, FD&C Blue No. 2. FD&C Green No. 3, Orange B, Citrus FD&C Red No. 2, FD&C Red No. 2, FD&C Red No. 3, FD&C Red No. 40, FD&C Yellow No. 5 and FD&C Yellow No. 6. The colorant(s) are preferably incorporated in the compositions at a concentration of 0 to 5% by weight and more preferably 0.5 to 2.5% by weight.

### Methods

In another embodiment, a method of enterally administering a pharmaceutical composition to postnatal swine is provided that comprises obtaining a swine pharmaceutical delivery composition, applying the composition to at least one treated pig in the vicinity of at least one piglet, and allowing the at least one piglet to voluntarily consume the composition from the at least one treated pig, wherein the at least one piglet exhibits a change in health not observed in a piglet that has not voluntarily consumed the composition.

The method comprises obtaining a swine pharmaceutical delivery composition, which may be prepared by adding and mixing the ingredients of the composition in a suitable vessel such as a stainless steel tank provided with a mixer. In a preferred aspect, all ingredients except for the active agent(s) are mixed with water or other suitable solvent to the desired viscosity, that once obtained is followed by addition of the active agent(s). All the ingredients are mixed again to form a final homogenous dispersion/solution for topical application.

The method comprises applying a composition to at least one pig (“treated pig”), which is preferably the piglet's mother, but may be another sow or other piglets or a combination thereof. The compositions are applied to the treated pig in an amount and for a period of time sufficient to attract a piglet that voluntarily consumes the composition.

In some aspects, the amount of the composition (in milliliters) that is applied is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, or about 250 mls or more. The amount of the composition that is applied may be inversely proportional to the concentration and/or potency of the active agent, and may depend on the particular application and dosing schedule. For example, if the composition is applied to a sow's teats, the targeted recipients may be multiple offspring of that sow in which case enough of the composition is applied such that an adequate dose of active agent may be consumed by each piglet.

In some aspects the compositions are applied once daily, once a week, twice a week, three times per week, biweekly, monthly, or bimonthly, etc. A single application at birth or soon after (e.g., between postnatal day 0 to 3) is generally sufficient. Another preferred application schedule is a first application between postnatal day 0 to 3 followed by a second application about two weeks after birth (approximately postnatal day 12 to 16).

Application may be through any suitable method of application and includes spraying, pouring, painting, and/or dripping. A preferred aspect is to provide a composition having a viscosity suitable for topical application by spraying, although compositions having a viscosity that are not conducive to spraying may be applied by pouring, painting, pouring or dripping.

The compositions are applied for a duration and frequency sufficient to elicit a change in health, growth, and/or feed conversion rate (FCR) of the consuming piglet less than about 1 month old (about postnatal day 30). In one aspect, the consuming piglet's FCR is greater than 2.5, 2.6, 2.8, 2.9, 3.0, 3.1, 3.2 or higher. In another aspect, the FCR of a voluntarily consuming piglet (individually or a population) is at least 5% greater than a piglet that has not voluntarily consumed the composition.

In another embodiment, a method of preparing a swine pharmaceutical delivery composition, the method comprising preparing an admixture comprising an adhesion enhancing agent, a pH adjusting agent, and a stabilizer; mixing the admixture with a solvent to form a mixture; adding at least one active agent to the mixture; and mixing the mixture to form a composition.

In some aspects the admixture is dry and powdered. In some aspects the solvent is water. In a preferred aspect the mixture is viscous. In some aspects the method further comprises adding a flavoring agent, a colorant, or both, to the mixture. In some aspects, the adhesion enhancing agent is polyvinyl pyrrolidone.

In some aspects, the at least one active agent is selected from the group consisting of a vitamin, mineral, amino acid, vaccine, and probiotic. In another aspect, the active agent comprises an amino acid, peptide, polypeptide, protein, enzyme, carbohydrate, lipid, or mineral, or combination thereof. The active agent is preferably incorporated in the compositions at a concentration of 0.0001% to 20% by weight and more preferably 0.0001% to 5% by weight.

The active agent may also be selected from the group consisting of biologically viable material, a food or feed ingredient, an antimicrobial agent, an antibiotic replacement agent, a prebiotic, a probiotic, and a pharmaceutical compound.

In some aspects, the pharmaceutical compound may be selected from the group consisting of analgesic, respiratory stimulant, mucolytic, and expectorant.

The pharmaceutically acceptable carrier may be selected from the group consisting of stabilizers, pH adjusting compositions, adjuvants, antimicrobial agents, anesthetics, corticosteroids, or a combination thereof.

In some aspects of the invention, the compositions may comprise a pharmaceutically acceptable carrier in a final concentration (v/v) of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, or any ranges therebetween including, for example, about 5% to about 10%, about 10% to about 15%, about 12% to about 13%, about 15% to about 20%, about 20% to about 25%, about 25% to about 30%, about 30% to about 35%, about 35% to about 40%, about 40% to about 45%, and about 45% to about 50%. In another aspect, the final concentration of the pharmaceutically acceptable carrier is about 12.5% v/v.

In another aspect the composition comprises an adhesion enhancing agent. In some aspects, the adhesion enhancing agent in a final concentration (w/v) of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, or any ranges therebetween including, for example, from about 0.5% to 15% w/v, from about 0.5% to 10% w/v, from about 0.5% to 5% w/v, from about 0.5% to 2% w/v. In another aspect, the final concentration of the adhesion enhancing agent is about 1.3%.

In some aspects the adhesion enhancing agent is a hydrophilic polymer or copolymer that is linear or branched, crosslinked, is not biodegradable. Suitable adhesion enhancing agents include polyvinylpyrrolidone, polyvinylpyrrolidone/vinyl acetate copolymers, waxes, mineral oil, plastigel (a blend of mineral oil and polyethylene), petrolatum, white petrolatum, shellac, versagel (blend of liquid paraffin, butene/ethylene/styrene hydrogenated copolymer), polyethylene waxes, microcrystalline waxes, polyisobutene, polyvinylpyrrolidone/vinyl acetate copolymers, and insoluble polyacrylate copolymers, xanthan, guar, pectins, gums, guar derivatives, chitosan, dextran, maltodextrin, carrageenans, starch, polyethylene glycol, albumin, cellulose ethers, hyaluronic acid, carboxymethylhydroxyethylcellulose, hydroxypropyl cellulose, gelatins, vinyl acetates, polyvinyl pyrrolidone-vinyl acetate copolymers, polyvinyl alcohols, polyphosphoesters, N-(2-hydroxypropyl) methacrylamide (HPMA) copolymers, polyacrylic acids, polyacrylamides, polyoxazolines, divinyl ether-maleic anhydride, polyphosphazenes, including derivatives and substitutions, and combinations thereof.

One or more of hydroxypropylmethyl cellulose (HPMC), hydroxypropyl cellulose (HPC), hydroxyethyl cellulose (HEC), and carboxy methyl cellulose (CMC), and salts thereof, are also suitable adhesion enhancing agents.

In some aspects, the adhesion enhancing agent has a molecular weight of 24,000 to 90,000.

In some aspects, the compositions comprising an adhesion enhancing agent are viscous. In further aspects the compositions have a viscosity (cPs) of at least about 10, 20, 30, 40, 50, 60, 70, 80, 90, 100, 150, 200, 250, 300, 350, 400, 450, 500, 550, 600, 650, 700, 750, 800 cPs or higher.

In additional aspects, the active agent used in the compositions comprising an adhesion enhancing agent comprises an ingredient selected from the group consisting of vitamin, mineral, amino acid, vaccine, and probiotic. In further aspects, the active agent comprises an amino acid, peptide, polypeptide, protein, enzyme, carbohydrate, lipid, or mineral, or combination thereof. In another aspect, the active agent is selected from the group consisting of biologically viable material, a food or feed ingredient, an antimicrobial agent, an antibiotic replacement agent, a prebiotic, a probiotic, and a pharmaceutical compound. In some aspects, the pharmaceutical compound may be selected from the group consisting of analgesic, respiratory stimulant, mucolytic, and expectorant.

In an embodiment where the composition comprises a flavoring agent, the agent is selected from nutritive and non-nutritive sweeteners, flavor additives, by-products and alternative ingredients. Some preferred flavoring agents include sucrose, glucose, sodium saccharin, sodium cyclamate, xylitol, perillartien, sucralose, D-tryptophan, aspartame, dihydrochalcones and the like, artificial fruit flavoring (e.g., strawberry flavoring), plasma protein (e.g., spray-dried plasma protein), cheese and cheese-like flavorings, dried milk, chocolate and chocolate by-products. The flavoring agent is preferably incorporated in the compositions at a concentration of 0% to 2% by weight and more preferably 0.1% to 0.5% by weight.

In an embodiment where the composition comprises a colorant, a colorant such as a pigment or dye or a combination thereof may be used. Suitable colorants include, but are not limited to, FD&C colorants such as FD&C Blue No. 1, FD&C Blue No. 2, FD&C Green No. 3, Orange B. Citrus FD&C Red No. 2, FD&C Red No. 2, FD&C Red No. 3, FD&C Red No. 40, FD&C Yellow No. 5 and FD&C Yellow No. 6. The colorant(s) are preferably incorporated in the compositions at a concentration of 0 to 5% by weight and more preferably 0.5 to 2.5% by weight.

In another embodiment, a kit for preparing a swine pharmaceutical delivery composition comprising a container comprising a swine pharmaceutical delivery composition and instructions for use is provided. In some aspects, the kit further comprises a flavoring agent and in some aspects it also comprises a colorant. The kit optionally includes a set of instructions containing dosage and administration information instructing a user regarding amounts of active ingredient(s) to add and mixing instructions.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Those of skill in the art should, in light of the present disclosure, will appreciate that many changes can be made in the specific embodiments which are disclosed and still obtain a like or similar result without departing from the spirit and scope of the invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight temperature is in degrees Centigrade, and pressure is at or near atmospheric.

### Example 1

Formula Composition to be added to water at 100 grams per 2-gallons of spray solution (range 50-200 grams per 2-gallons).

| | Dry | Diluted ready to spray gel | |
|---|---|---|---|
| | admixed | (example 100 grams of | |
| Ingredient | powder | powder per 2-gal spray) | |
| Maltodextrin | 33.888% | 16.944 | g/gal |
| Carboxymethyl cellulose | 40.000% | 10.000 | g/gal |
| Sodium chloride | 16.720% | 8.360 | g/gal |
| Certificated food coloring | 5.000% | 2.500 | g/gal |
| Disodium phosphate | 1.638% | 0.819 | g/gal |
| Artificial flavoring | 1.000% | 0.500 | g/gal |
| Sodium thiosulfate | 1.000% | 0.500 | g/gal |
| Artificial sweetener | 0.500% | 0.250 | g/gal |
| Potassium chloride | 0.102% | 0.051 | g/gal |
| L-lysine monohydrochloride | 0.076% | 0.038 | g/gal |
| Sodium bicarbonate | 0.076% | 0.038 | g/gal |

| | | |
|---|---|---|
| Water | — | QS to 1 gallon |

### Example 2

Formula Composition to be added to water at 200 grams per 2-gallons of spray solution (range 100-400 grams per 2-gallons).

| | Dry | Diluted ready to spray gel | |
|---|---|---|---|
| | admixed | (example 200 grams of | |
| Ingredient | powder | powder per 2-gal of spray) | |
| Polyvinylpyrrolidone | 50.000% | 50.000 | g/gal |
| Maltodextrin | 16.944% | 16.944 | g/gal |
| Carboxymethyl cellulose | 20.000% | 20.000 | g/gal |
| Sodium chloride | 8.360% | 8.360 | g/gal |
| Certificated food coloring | 2.500% | 2.500 | g/gal |
| Disodium phosphate | 0.819% | 0.819 | g/gal |
| Artificial flavoring | 0.500% | 0.500 | g/gal |
| Sodium thiosulfate | 0.500% | 0.500 | g/gal |
| Artificial sweetener | 0.250% | 0.250 | g/gal |
| Potassium chloride | 0.051% | 0.051 | g/gal |
| L-lysine monohydrochloride | 0.038% | 0.038 | g/gal |
| Sodium bicarbonate | 0.038% | 0.038 | g/gal |

| | | |
|---|---|---|
| Water | — | QS to 1 gallon |

### Example 3

Method of preparing a composition and application. The formula of Example 2 is prepared and the solution is thoroughly mixed. The composition is then strained to remove lumps which are larger than the strainer openings. A desired active ingredient is added in the solution at the desired dosage. Active ingredients required to dose one litter of piglets is mixed into 125 mls of solution. The solution is sprayed uniformly on a sow's underline while she is standing.

### Example 4

To assess the ability of the inventive compositions to stabilize and therefore improve the efficacy of vaccines, an in vitro analysis was performed using a avirulent live vaccine against Erysipelothrix rhusiopathiae. Bacterial plate counts of treated versus sterile water and chlorinated water controls. Chlorinated water was used to model water sources on farms which typically have elevated chlorine levels. Samples were taken from the different treatments over a four-hour period to represent the vaccine manufacturer's maximum recommended time in solution. The efficacy of the vaccine stabilizing composition shown in Table 1 was determined by comparing bacterial plate counts of water treated with the composition of Table 1 versus untreated sterile water and chlorinated water.

Formula composition to be added to water at 100 grams per 2-gallons of spray solution.

TABLE 1

| Ingredient Name | Amount (g) | Pet |
|---|---|---|
| TIC GUM CMC 2500C (50#) | 800.0000 | 40.00000 |
| CLINTOSE 18 CR 18 MALTODEXTRI | 749.4320 | 37.47160 |
| SALT, MORTON TFC PUREX (50#) | 334.0000 | 16.70000 |
| DISODIUM PHOSPHATE 50# | 32.7600 | 1.63800 |
| DRY RED BERRY WS 18691 (LB) | 20.0000 | 1.00000 |
| SODIUM THIOSULFATE ANHYD 50# | 20.0000 | 1.00000 |
| L-1143 EMERALD GREEN (25#) | 20.0000 | 1.00000 |
| POWERSWEET 50# | 10.0000 | 0.50000 |
| SUPPLE-K KCL 50# POTASSIUM CH | 8.7280 | 0.43640 |
| MONOPOTASSIUM PHOSPHATE 50 | 2.0400 | 0.10200 |
| L-LYSINE HCL 55# | 1.5200 | 0.07600 |
| SODIUM BICARBONATE USP GRAD | 1.5200 | 0.07600 |
| PHOSPHORUS | 0.3806 | |
| POTASSIUM | 0.2582 | |
| SULPHUR | 0.6400 | |
| SODIUM | 7.3552 | |
| CHLORINE | 10.4088 | |
| BICARB | 0.0752 | |
| SALT | 16.7000 | |
| LYSINE | 0.0599 | |
| PROTEIN | 0.0717 | |

Vaccine was thawed in a cold water bath 45-60 minutes prior to use and a 1:10 serial dilution was performed. 9 ml of dH2O was pipetted into 8 sterile tubes. 1 ml of Ingelvac® ERY-ALC vaccine (per labeled dose) was transferred into tube 1, then transfer 0.5 ml of the mixture to a blood agar plate and spread. 1 ml of contents in tube 1 was transferred to tube 2, then 0.5 ml of the mixture was transferred to a blood agar plate and spread. 1:10 serial dilutions were continued and plated through tube 8. Plates were incubated at 37° C. for 24 hours. CFU counts were determined for plates with 25-250 colonies. Plates with more than 250 colonies were classified as TNTC, and plates with fewer than 25 colonies were classified as TFTC.

As noted previously, three treatments were used and each performed in triplicate. Ingelvac® ERY-ALC vaccine is presented in vials containing 1 label dose per ml. Vaccine was added to solutions at the rate of 1 label dose in 10 ml to replicate common gel delivery dilutions for pre-weaned pigs. Treatment 1 (sterile H20) contained only vaccine and sterile dH2O, attaining a total volume of 250 ml to represent a non-treated and non-chlorinated water source. Treatment 2 (chlorinated H₂0) contained the vaccine and 6% chlorine (sodium hypochlorite) with sterile dH₂O added to create a total volume of 250 ml. 25 mls were removed and replaced with 25 mls of vaccine added by pipette.

Treatment 2 represented a non-treated but chlorinated water source (similar to tap water) having a free chlorine concentration of 4 ppm (confirmed by using Fisher Scientific Test Strips with a range of 0-6 ppm). 25 mls were removed and replaced with 25 mls of vaccine added by pipette.

Lastly, Treatment 3 (composition of Table 1) was formed by adding 6.6 g of sterile water to 250 mls of the solution of Treatment 2. 25 mls were removed for total solution of 225 mls. Colorant was added, the mixture was homogenized to form the stabilizer composition and ensuring that the gel was free of lumps. 25 mls of vaccine was then pipetted and uniformly mixed into the gel.

Treatments 1, 2, and 3, were incubated at room temperature and sampled 0.5 ml at 0.0, 0.5, 2, and 4 hours. Samples were plated to determine CFU concentrations. The averages of each treatment on CFU concentrations of the live virus are shown in Table 2. As shown in Table 2, treatment with the vaccine stabilizer composition of Table 1 prevents the breakdown of the live vaccine titer in water with high-chlorine content typical of farms.

TABLE 2

| | TREATMENT TIME | | | |
|---|---|---|---|---|
| CONDITION | 0 hr | 0.5 hr | 2 hr | 4 hr |
| Sterile H₂O | 10 × 10⁸ | 3.9 × 10⁸ | 1.5 × 10⁸ | 2.2 × 50⁸ |
| Chlorinated H₂O | 7.3 × 10³ | TFTC | TFTC | TFTC |
| Treatment | 9.1 × 10⁸ | 9.8 × 10⁸ | 6.7 × 10⁸ | 9.4 × 10⁸ |
| (composition of Table 1) | | | | |

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present invention.

1. A swine pharmaceutical delivery composition comprising an active agent and a pharmaceutically acceptable carrier.

2. The composition of claim 1, wherein the pharmaceutically acceptable carrier is selected from the group consisting of preservatives, stabilizers, pH adjusting compositions, adjuvants, alcohols, glycols, glycerols, and glycerines, lanolin and derivatives thereof, fatty acids and derivatives thereof, fatty alcohols and derivatives thereof, and fatty esters and derivatives thereof, or combinations thereof.

3. The composition of claim 1, wherein the composition further comprises an adhesion enhancing agent.

4. The composition of claim 3, wherein the adhesion enhancing agent is at a final concentration from about 0.5% to 15% w/v.

5. The composition of claim 3, wherein the adhesion enhancing agent is at a final concentration from about 0.5% to 10% w/v.

6. The composition of claim 3, wherein the adhesion enhancing agent is at a final concentration from about 0.5% to 5% w/v.

7. The composition of claim 3, wherein the adhesion enhancing agent is at a final concentration from about 0.5% to 2% w/v.

8. The composition of claim 3, wherein the adhesion enhancing agent is at a final concentration of about 1.3%.

9. The composition of claim 3, wherein the adhesion enhancing agent is polyvinyl pyrrolidone or copolymers thereof.

10. The composition of claim 9, wherein the adhesion enhancing agent has a molecular weight of 24,000 to 90,000.

11. The composition of claim 10, wherein the adhesion enhancing agent is a hydrophilic polymer or copolymer.

12. The composition of claim 11, wherein the hydrophilic polymer or copolymer is linear or branched.

13. The composition of claim 11, wherein the hydrophilic polymer or copolymer is crosslinked.

14. The composition of claim 11, wherein the hydrophilic polymer or copolymer is not biodegradable.

15. The composition of claim 11, wherein the hydrophilic polymer or copolymer is selected from the group consisting of xanthan, guar, pectins, gums, guar derivatives, chitosan, dextran, maltodextrin, carrageenans, starch, polyethylene glycol, albumin, cellulose ethers, hyaluronic acid, carboxymethylhydroxyethyl cellulose, hydroxypropyl cellulose, gelatins, vinyl acetates, polyvinyl pyrrolidone-vinyl acetate copolymers, polyvinyl alcohols, polyphosphoesters, N-(2-hydroxypropyl) methacrylamide (HPMA) copolymers, polyacrylic acids, polyacrylamides, polyoxazolines, divinyl ether-maleic anhydride copolymer, methylvinyl ether-maleic anhydride copolymer, polyphosphazenes, polyethylene oxide, carbomers, including derivatives and substitutions, and combinations thereof.

16. The composition of claim 15, wherein the cellulose ether comprises one or more of hydroxypropylmethyl cellulose, hydroxypropyl cellulose, hydroxyethyl cellulose, and carboxy methyl cellulose, and salts thereof.

17. The composition of claim 1, wherein the active agent comprises an ingredient selected from the group consisting of vitamin, mineral, amino acid, vaccine, and probiotic.

18. The composition of claim 1, wherein the active agent comprises an amino acid, peptide, polypeptide, protein, enzyme, carbohydrate, lipid, or hormone, or combination thereof.

19. The composition of claim 1, wherein the active agent is selected from the group consisting of biologically viable material, a food or feed ingredient, an antimicrobial agent, an antibiotic replacement agent, a prebiotic, a probiotic, and a pharmaceutical compound.

20. The composition of claim 19, wherein the pharmaceutical compound is selected from the group consisting of analgesic, respiratory stimulant, mucolytic, and expectorant.

21. A method of enterally administering a pharmaceutical composition to postnatal swine, the method comprising:
obtaining the composition of claim 3;
applying the composition to at least one treated pig in the vicinity of at least one piglet; and
allowing the at least one piglet to voluntarily consume the composition from the at least one treated pig,
wherein the at least one piglet exhibits a change in health not observed in a piglet that has not voluntarily enterally received the composition.

22. The method of claim 21, wherein the step of applying comprises topically applying the composition.

23. The method of claim 22, wherein the composition is topically applied by spraying, pouring, or dripping.

24. The method of claim 22, wherein the composition is topically applied to at least one teat of at least one treated pig.

25. The method of claim 21, wherein the composition comprises a vaccine.

26. The method of claim 25, wherein the change in health is a change in live viral or bacterial titer.

27. The method of claim 26, wherein the change in health is a decrease in live viral or bacterial titer.

28. The method of claim 27, wherein the decrease in live viral or bacterial titer is detected after 2 weeks after application.

29. The method of claim 21, wherein the composition further comprises a colorant.

30. The method of claim 21, wherein the composition further comprises a flavor agent.

31. The method of claim 21, wherein the composition comprises a colorant and a flavor agent.

32. A method of preparing a swine pharmaceutical delivery composition, the method comprising:
preparing an admixture comprising an adhesion enhancing agent, a pH adjusting agent, and a stabilizer;
mixing the admixture with a solvent to form a mixture;
adding at least one active agent to the mixture; and
mixing the mixture to form a composition.

33. The method of claim 32, wherein the admixture is dry and powdered.

34. The method of claim 32, wherein the solvent is water.

35. The method of claim 32, further comprising adding a flavoring agent, a colorant, or both, to the mixture.

36. The method of claim 32, wherein the adhesion enhancing agent is polyvinyl pyrrolidone.

37. The method of claim 32, wherein the at least one active agent is selected from the group consisting of a vitamin, mineral, amino acid, vaccine, and probiotic.

38. The method of claim 32, wherein the mixture is viscous.

39. A kit for preparing a swine pharmaceutical delivery composition comprising:
a container comprising the composition of claim 3, wherein the composition further comprises a flavoring agent, a colorant, or both; and
instructions for use.

40. (canceled)

41. (canceled)
